(19) United States
(12) Patent Application Publication
(10) Pub. No.: US 2026/0115700 A1
(43) Pub. Date: Apr. 30, 2026

(54) TANTALUM CATALYST COMPOSITION AND METHOD OF USING THE SAME

(71) Applicant: Battelle Memorial Institute, Richland, WA (US)

(72) Inventors: Vanessa Dagle, Richland, WA (US); Robert A. Dagle, Richland, WA (US); Martin Affandy, Kennewick, WA (US)

(73) Assignee: Battelle Memorial Institute, Richland, WA (US)

(21) Appl. No.: 18/929,391

(22) Filed: Oct. 28, 2024

(51) Int. Cl. B01J 23/847; B01J 21/08; B01J 23/20; B01J 23/86; B01J 37/02; B01J 37/08; C07C 1/24

(52) U.S. Cl. CPC B01J 23/8476; B01J 21/08; B01J 23/20; B01J 23/868; B01J 37/0205; B01J 37/08; C07C 1/24; C07C 2521/02; C07C 2521/06; C07C 2523/06; C07C 2523/10; C07C 2523/50; C07C 2523/72; C07C 2523/86

(57) ABSTRACT

Disclosed herein are aspects of a method for converting an oxygenate feedstock into an olefin-rich product. In some aspects, the method comprises exposing an oxygenate feedstock to a tantalum catalyst composition to form an olefin-rich product. In some aspects, the tantalum catalyst composition comprises tantalum and a support comprising (i) aluminum and/or silicon, and (ii) oxygen.

### ACKNOWLEDGMENT OF GOVERNMENT SUPPORT

This invention was made with government support under Contract DE-AC0576RL01830 awarded by the U.S. Department of Energy. The government has certain rights in the invention.

### FIELD

The present disclosure concerns a tantalum catalyst composition, methods of making the tantalum catalyst composition, and methods of using the tantalum catalyst composition.

### BACKGROUND

Petroleum depletion and environmental concerns have led to renewed interest in using biomass as a carbon source for the production of biofuels. Ethanol and ethanol-derived aldehyde conversion to biofuels represent an appealing bioenergy technology. Accordingly, ethanol and ethanol-derived aldehydes are commercially produced from renewable biomass or waste sources. Additionally, advancements in production efficiency and feedstock diversification may potentially lead to excess ethanol having competitive pricing for production of a wide range of fuels and/or commodity chemicals. Moreover, with the lightening of feedstocks to steam crackers, due to the increasing use of shale gas instead of naphtha as feed, there may be a shortage of C₃ and C₄ olefins as industrial starting materials. Thus, there is a need for methods of converting oxygenated feedstocks, such as ethanol, into olefins.

### SUMMARY

Disclosed embodiments of the present disclosure advantageously provide a tantalum catalyst composition for producing an olefin-rich product from an oxygenate feedstock with greater selectivity and thermal stability than Zr-based catalysts. Certain disclosed aspects concern a method, comprising: exposing an oxygenate feedstock to a tantalum catalyst composition to form an olefin-rich product, the tantalum catalyst composition comprising a support comprising (i) aluminum and/or silicon, and (ii) oxygen.

Certain disclosed aspects concern a method of making a tantalum catalyst composition for converting an oxygenate feedstock into an olefin-rich product. In some aspects, the method comprises impregnating a support comprising (i) aluminum and/or silicon, and (ii) oxygen, with a tantalum precursor to form a catalyst precursor.

Certain disclosed aspects concern a tantalum catalyst composition for converting an oxygenate feedstock into an olefin-rich product, the tantalum catalyst composition comprising: a tantalum oxide in an amount ranging from 1% to 50% based on total weight of the tantalum catalyst composition; and a support comprising aluminum and/or silicon.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 shows a diagram of a system for producing an olefin-rich product from an oxygenate feedstock according to aspects of the present disclosure.

FIG. 2 shows a diagram of a system for producing an olefin-rich product from an oxygenate feedstock according to additional aspects of the present disclosure.

FIG. 3 shows a diagram of a system for producing an olefin-rich product from an oxygenate feedstock according to yet additional aspects of the present disclosure.

FIG. 4 shows a flow diagram of an exemplary method for making a tantalum catalyst composition for converting an oxygenate feedstock into an olefin-rich product.

FIG. 5 shows a flow diagram of an exemplary method for producing an olefin-rich product from an oxygenate feedstock.

FIG. 6 shows a plot of conversion and selectivity toward total olefins, acetaldehyde, and ethylene using 4% Cu/6% Ta₂O₅ supported on silicon-containing materials.

FIG. 7 shows a plot of conversion and selectivity toward total olefins, acetaldehyde, and C₂-C₅ alkanes using 4% Cu/6% Ta₂O₅ supported on SBA-16 silica.

FIG. 8 shows a plot of conversion and selectivity toward total olefins, acetaldehyde, and C₂-C₅ alkanes using 4% Cu/6% Ta₂O₅ supported on DAVISIL® 646 silica gel.

FIG. 9 shows a plot of conversion and selectivity toward ethylene, propene, butene, and C₅₊ olefins using 4% Cu/6% Ta₂O₅ supported on DAVISIL® 646 silica gel.

FIG. 10 shows a plot of conversion and selectivity over time toward total olefins, acetaldehyde, and C₂-C₅ alkanes using two catalyst compositions according to aspects of the present disclosure.

FIG. 11 shows a plot of conversion and selectivity toward total olefins, acetaldehyde, and C₂-C₅ alkanes using different ethanol/water ratios.

FIG. 12 shows a plot of conversion and selectivity over time toward total olefins, acetaldehyde, and C₂-C₅ alkanes using a 0.2% Cr/4% Cu/20% Ta₂O₅ catalyst composition supported on DAVISIL® 646 silica gel.

FIG. 13 shows a plot of conversion and selectivity for a single catalyst material and for a combination of catalyst materials according to aspects of the present disclosure.

FIG. 14 shows a plot of conversion and selectivity towards total olefins, total oxygenates, acetaldehyde, DEE, butanol, and other oxygenates using a 4% Cu/6% Ta₂O₅ catalyst material and for a 4% Cu/6% ZrO₂ catalyst material according to aspects of the disclosure.

### DETAILED DESCRIPTION

### I. Abbreviations

Ag: silver
BD: butadiene
CO₂: carbon dioxide
Cr: chromium
Cu: copper
Cu(NO₃)₂·XH₂O: copper (II) nitrate hydrate
Cₓ material: a compound comprising x carbon atoms, where x is an integer.
DEE: diethyl ether
H₂: hydrogen
hr: hour
g: gram
m: meter
N₂: nitrogen
NO: nitric oxide
Pa: Pascal
psig: pound per square inch (gauge)
SiO₂: silicon dioxide
Ta₂O₅: tantalum (V) oxide
Ta₂(OC₂H₅)₁₀: tantalum (V) ethoxide
TOS: time on stream
WHSV: weight hourly space velocity
Zn: zinc
Zr: zirconium
ZrO₂: zirconium oxide

### II. Overview of Terms, Ranges, and Definitions

Both the foregoing general description and the following detailed description are exemplary and explanatory only and are not intended to limit the scope of the present disclosure.

As used herein, the use of the singular includes the plural unless specifically stated otherwise. For example, the singular forms “a”, “an” and “the” as used in the specification also include plural aspects unless the context dictates otherwise. Similarly, any singular term used in the specification also means plural or vice versa, unless the context dictates otherwise.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Unless explained otherwise, method steps represented by dashed boxes and/or lines in flow diagrams are optional. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

The term “or combinations thereof” as used herein refers to all permutations and combinations of the listed items preceding the term. For example, “A, B, C, or combinations thereof” is intended to include at least: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth.

The term “alcohol” generally refers to an organic compound including at least one hydroxyl group. Alcohols may be monohydric (including one —OH group), dihydric (including two —OH groups; diols, such as glycols), trihydric (including three-OH; triols, such as glycerol) groups, or polyhydric (including three or more —OH groups; polyols). The organic portion of the alcohol may be aliphatic, cycloaliphatic (alicyclic), heteroaliphatic, cycloheteroaliphatic (heterocyclic), polycyclic, aryl, or heteroaryl, and may be substituted or unsubstituted unless otherwise specified. Ethanol, butanol, and crotyl alcohol are examples of alcohols.

The term “aldehyde” generally refers to a carbonyl-bearing functional group or a compound bearing said functional group, said functional group having a formula

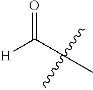 [SMILES: *C=O]

where the line drawn through the bond indicates that the functional group can be attached to any other moiety, but that such moiety simply is not indicated. Acetaldehyde, butyraldehyde, and crotonaldehyde are examples of aldehydes.

The term “C₂-C₄ compound” generally refers to a compound comprising 2 to 4 carbon atoms (such as 2, 3, or 4 carbon atoms). Ethanol is an example of a C₂ alcohol.

The term “calcination” generally refers to a process of heating a solid to a temperature below its melting point to bring about a state of thermal decomposition or a phase transition other than melting. Calcination can include: 1) thermal dissociation, including destruction of organic compounds, e.g., loss of carbon dioxide from limestone, 2) removal of crystalline waters of hydration, 3) decomposition of volatile organic matter, or 4) phase transformations, such as devitrification of glass. Additionally, calcination may cause oxidation of some metals.

The term “carboxylic acid” generally refers to a carbonyl-bearing functional group having a formula RCOOH or a compound containing such a functional group, where R is aliphatic, heteroaliphatic, such as alkyl or heteroalkyl. Ethyl acetate is an example of a carboxylic acid.

The term “catalyst composition” generally refers to a substance, usually present in small amounts relative to reactants, which increases the rate of a chemical reaction without itself being consumed or undergoing a chemical change. A catalyst composition also may enable a reaction to proceed under different conditions (e.g., at a lower temperature) than otherwise possible. Catalyst compositions typically are highly specific with respect to the reactions in which they participate. The term “tantalum catalyst composition” generally refers to a tantalum catalyst composition that comprises a tantalum species (e.g., a tantalum ion).

The term “catalyst bed” generally refers to a layer of a tantalum catalyst composition placed within a reactor through which reactants pass and undergo a chemical reaction. Catalyst beds can be configured in various ways, such as fixed beds, fluidized beds, or packed beds, depending on the specific application and desired reaction condition. A mixed catalyst bed refers to a catalyst bed that contains a mixture of different types of catalysts. This can be done to optimize the reaction by combining catalysts that have different properties or functionalities. For example, one catalyst might be highly active but less selective, while another might be more selective but less active.

The term “conversion” generally refers to an amount of reactants that are converted into a product during a chemical reaction. The conversion rate can indicate the efficiency and effectiveness of a catalytic process.

The term “ester” generally refers to a chemical compound derived from an organic acid (general formula: RCO₂H) where the hydrogen of the —OH (hydroxyl) group is replaced by, e.g., an aliphatic (e.g., alkyl) or aromatic (e.g., aryl or heteroaryl) group. A general formula for an ester derived from an organic acid is shown below:

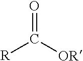

where R and R′ denote a group selected from, for example, aliphatic, aryl, heteroaliphatic, heteroaryl, etc.

The term “ether” generally refers to a class of organic compounds containing an ether group, that is an oxygen atom connected to two aliphatic and/or aromatic (e.g., aryl) groups, and having a general formula R—O—R′, where R and R′ may be the same or different. Diethyl ether is an example of an ether.

The term “impregnation” generally refers to a process by which a tantalum precursor and/or a dopant are introduced into a support material to form a tantalum catalyst composition or a catalyst precursor. Impregnation can refer to co-impregnation or sequential impregnation. Some examples of impregnation methods include incipient wetness impregnation, precipitation (including co-precipitation or sequential precipitation), and sol-gel methods.

The term “intermediate composition” generally refers to a mixture of reactants, products, and possibly intermediates that exist in the space between two sequential catalyst beds within a reactor or between two sequential reactors.

The term “ketone” generally refers to a carbonyl-bearing substituent having a formula

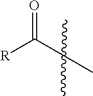

wherein each R independently typically is a group selected from, for example, aliphatic, substituted aliphatic, aryl, arylalkyl, heteroaryl, etc.

The term “metal dopant” generally refers to a metal added to a tantalum catalyst composition to alter one or more of the tantalum catalyst composition's physical and/or chemical properties.

The term “olefin” generally refers to an unsaturated aliphatic hydrocarbon having at least one double bond. Ethylene is an example of an olefin.

The term “olefin-rich product” generally refers to a product resulting from a reaction between a feedstock and a tantalum catalyst composition according to aspects of the present disclosure, and which contains a greater amount and/or a greater concentration of olefins than the feedstock.

The term “oxygenate feedstock” generally refers to a starting material for a chemical process that contains at least one oxygen atom.

The term “selectivity” generally refers to ability of a catalyst to direct a reaction to preferentially form a particular product. For example, suppose a catalyst can dehydrate compound A to form either compound B, compound C, or a mixture of compounds B and C. In this exemplary illustration, if the catalyst has a compound B selectivity of 90%, compound A will be dehydrated to form 90% compound B and 10% compound C. Total olefin selectivity refers to selectivity for all olefin products.

The term “support” generally refers to a material that provides a mechanical/physical foundation for the tantalum catalyst composition. In some aspects, the support can participate in chemical reactions along with the tantalum catalyst composition.

The term “tantalum oxide” generally refers to an oxidized tantalum compound comprising tantalum and oxygen. Ta₂O₅ is an example of a tantalum oxide.

The term “transition metal” generally refers to an element whose atoms have an incomplete d-shell or can give rise to cations with an incomplete d-shell. Transition metals are found in Groups 3 through 12 on the periodic table and include elements such as iron, copper, and gold.

While the present teachings are described in conjunction with various aspects of the disclosure, it is not intended that the present teachings be limited to such aspects. On the contrary, the present teachings encompass various alternatives, modifications, and equivalents, as will be appreciated by a person of ordinary skill in the art in light of the present teachings, unless otherwise indicated.

### III. Introduction

Olefins, such as butenes, can be valuable chemical commodities. For example, olefins may be found in industrial applications, such as producing hydrocarbon fuels (e.g., gasoline, jet fuel, or diesel). In some instances, ethanol can be converted into butene over Zr-based catalysts, such as Ag/ZrO₂/SiO₂ and Cu/ZrO₂/SiO₂. However, these Zr-based catalysts suffer from loss of conversion over time.

To address these issues and other drawbacks associated with conventional catalyst compositions, the present disclosure provides a tantalum catalyst composition for converting an oxygenate feedstock into an olefin-rich product. In some aspects, the tantalum catalyst composition comprises an acidic metal component, such as tantalum. In some aspects, the tantalum catalyst composition further comprises a support comprising (i) aluminum and/or silicon, and (ii) oxygen. The tantalum catalyst composition can, in some aspects, catalyze conversion of an oxygenate feedstock into an olefin-rich product with greater selectivity, thermal stability, and longevity than Zr-based catalysts.

### IV. Tantalum Catalyst Composition, Method, and System

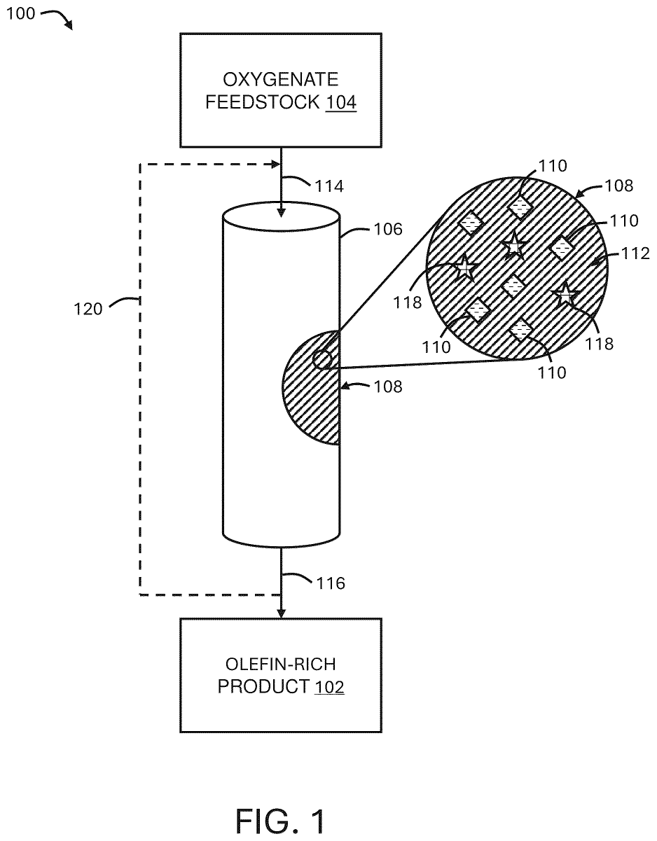

FIG. 1 shows a simplified diagram of a system 100 for producing an olefin-rich product 102 from an oxygenate feedstock 104 according to aspects of the present disclosure. The system 100 comprises a reactor 106 that houses a tantalum catalyst composition 108. In some aspects, the reactor 106 comprises a fixed-bed reactor. It will also be appreciated that any other suitable reactor can be used. Other examples of suitable reactors include fluidized bed reactors, circulating fluidized bed reactors, packed bed reactors, tubular reactors, trickle-bed reactors, and batch reactors.

The tantalum catalyst composition 108 comprises a metal component 110 and a support material 112. In some aspects, the metal component 110 comprises tantalum. In some aspects, the tantalum takes the form of a tantalum oxide (e.g., Ta₂O₅). The tantalum oxide can, in some aspects, acidify the tantalum catalyst composition 108 (for example, by making the tantalum catalyst composition 108 more oxidative). This can increase the catalytic activity of the tantalum catalyst composition 108 above that of the support material 112.

In some aspects, the metal component 110 is present in an amount ranging from 1 wt. % to 50 wt. % (based on total weight of the tantalum catalyst composition 108). In some aspects, the metal component 110 is present in an amount ranging from 1 wt. % to 20 wt. % based on total weight of the tantalum catalyst composition. In some aspects, the metal component 110 is present in an amount ranging from 1.5 wt. % to 18 wt. % (based on total weight of the tantalum catalyst composition). As described in more detail below, in some aspects, increasing the amount of the metal component relative to the overall weight of the tantalum catalyst composition can increase the conversion rate of the tantalum catalyst composition and total olefin selectivity of the tantalum catalyst composition.

As introduced above, the tantalum catalyst composition 108 further comprises support material 112. In some aspects, the support material 112 is present in an amount ranging from 10 wt. % to 90 wt. % based on total weight of the tantalum catalyst composition 108. In some aspects, the support material 112 is present in an amount ranging from 50 wt. % to 75 wt. % of the tantalum catalyst composition 108.

In some aspects, the support material 112 comprises (i) aluminum and/or silicon, and (ii) oxygen. For example, in some aspects, the support material 112 can comprise silica (e.g., SiO₂). In some aspects, silica can serve as an acidic component, which can contribute to the catalytic activity of the tantalum catalyst composition 108. Other examples of suitable support materials include zeolites, carbon, alumina (e.g., Al₂O₃), and combinations thereof (with or without silica).

Some examples of suitable materials for the support material 112 include a mesoporous silica material (e.g., SBA-15 mesoporous silica or SBA-16 mesoporous silica), a silica gel composition (e.g., DAVISIL® 646 provided by W.R. Grace & Co., DAVICAT® 57 provided by W.R. Grace & Co., DAVICAT® 59 provided by W.R. Grace & Co., DAISOGEL® SP-100-50 provided by DAISO® Fine Chem USA, Inc., or silica gel that conforms to GOST standard no. 3956-76), an aluminosilicate material (e.g., MS-13 silica/alumina), a zeolite material (e.g., dealuminated zeolite), or any combination thereof.

In some aspects, the support material 112 has a surface area ranging from 10 to 1000 m²/g. In some aspects, the support material 112 has a surface area ranging from 20 to 500 m²/g. In some aspects, the support material 112 has a surface area ranging from 250 to 500 m²/g. In some certain aspects, the support material 112 has a surface area ranging from 30 to 50 m²/g. In other certain aspects, the surface area can be greater than 1000 m²/g. In some aspects, providing a larger surface area can increase reactivity relative to a catalyst material with a smaller surface area. However, those in the art will recognize that certain levels of large surface areas can lead to structural instability, and the rate of the reaction can be constrained by mass transport (e.g., a rate of diffusion of a solvent through the support material), and thus will recognize surface area maxima with the benefit of the present disclosure.

In some aspects, the support material 112 is porous. In some aspects, the support material 112 has a pore diameter ranging from 10 to 200 Angstroms. In some aspects, the support material 112 has a pore diameter ranging from 20 to 100 Angstroms. In some particular aspects, the support material 112 has a pore diameter ranging from 30 to 50 Angstroms. In some aspects, numerous pores having smaller pore diameters can provide greater surface area than larger pores. However those in the art will recognize that certain smaller sizes of pores can restrict mass transport and certain larger sizes of pores can impact structural stability, and will thus recognize other suitable pore size minima and maxima with the benefit of the present disclosure.

In some aspects, the system 100 can also include a reactor inlet 114 and/or a reactor outlet 116. In some aspects, the reactor inlet 114 can be coupled to both the reactor 106 and at least one reactor reservoir, such as for example, a feed from a pump that includes the oxygenate feedstock 104.

In some aspects, the oxygenate feedstock 104 comprises a compound having two to four carbon atoms and at least one oxygen atom. Some examples of suitable oxygenate feedstock materials include, for example, an alcohol, an ether, an aldehyde, a ketone, a carboxylic acid, an ester, or a combination thereof. In some aspects, the oxygenate feedstock comprises ethanol, acetaldehyde, ethyl acetate, diethyl ether, butyraldehyde, butanol, crotonaldehyde, crotyl alcohol or a combination thereof. In some aspects, and as described in more detail below in the examples section, the oxygenate feedstock can be mixed with water. In some aspects, the oxygenate feedstock is in a solution or a mixture that has a water content of 5 wt. % to 70 wt. %. In some aspects, the water content ranges from 5 wt. % to 50 wt. %. In some aspects, the water content ranges from 5 wt. % to 25 wt. %.

In some aspects, the tantalum catalyst composition 108 further comprises at least one metal dopant 118. In some aspects, the metal dopant 118 comprises a Group 6, Group 11, or Group 12 transition metal. In some aspects, the metal dopant 118 is selected from Cr, Zn, Cu, Ag, or a combination thereof. The presence of the metal dopant 118 can, in some aspects, increase the conversion rate relative to an undoped tantalum catalyst composition. As described in more detail below in the examples section, the selection of certain metal dopant(s) 118 can also, in some aspects, tailor selectivity towards one or more certain olefin products (e.g., ethylene).

The metal dopant 118 is, in some aspects, provided in an amount ranging from 0.25 wt. % to 30 wt. % based on total weight of the tantalum catalyst composition. In some aspects, the amount of the at least one metal dopant 118 ranges from 0.25 wt. % to 20 wt. % based on total weight of the tantalum catalyst composition. In some aspects, the amount of the at least one metal dopant 118 ranges from 1 wt. % to 16 wt. % based on total weight of the tantalum catalyst composition.

As described in more detail below, in some aspects, the olefin-rich product 102 comprises a C₂-C₅ olefin, such as ethylene, propylene, butene, or a combination thereof. In some aspects, the olefin-rich product 102 can additionally include an unreacted portion of the oxygenate feedstock 104, one or more byproducts formed from a reaction between the oxygenate feedstock 104 and the catalyst material 108, or a combination thereof. In some aspects, such byproducts can comprise one or more C₂-C₅ alkanes.

In some aspects, and as indicated at step 120 of FIG. 1, at least a portion of a process stream leaving the reactor at the reactor outlet 116 can be recycled to the reactor inlet 114. In this manner, the olefin-rich product can be further enriched with desired olefins by making one or more additional passes through the reactor 106.

In some aspects, the tantalum catalyst composition 108 demonstrates improved conversion rates relative to conventional zirconium-based catalysts systems. For example, in some aspects, a greater than 85% conversion of the oxygenate feedstock into the olefin-rich product can be obtained using the method and tantalum catalyst composition according to the present disclosure. Solely by way of example, and as described in more detail below, a 4% Cu/6% Ta₂O₅ catalyst material supported on MS-13 demonstrated nearly complete conversion, with over 80% selectivity for ethylene.

In some aspects, the overall conversion of the oxygenate feedstock 104 into one or more different products (e.g., one or more desired olefins, including any oxidized products, such as an aldehyde) is greater than or equal to 85%. In some aspects, at least 90% of the oxygenate feedstock 104 is converted into the one or more product(s). In some aspects, at least 95% of the oxygenate feedstock 104 is converted into the one or more product(s).

In some aspects, the selectivity of converting the oxygenate feedstock into desired olefin products (e.g., butenes, such as 1-butene, 2-butene, or combinations thereof) is greater than or equal to 50%. In some aspects, the selectivity of the conversion is greater than or equal to 60%, favoring olefin products like butenes. In some aspects, the selectivity of the conversion is greater than or equal to 70%, favoring olefin products like butenes.

### V. Second Catalyst Composition

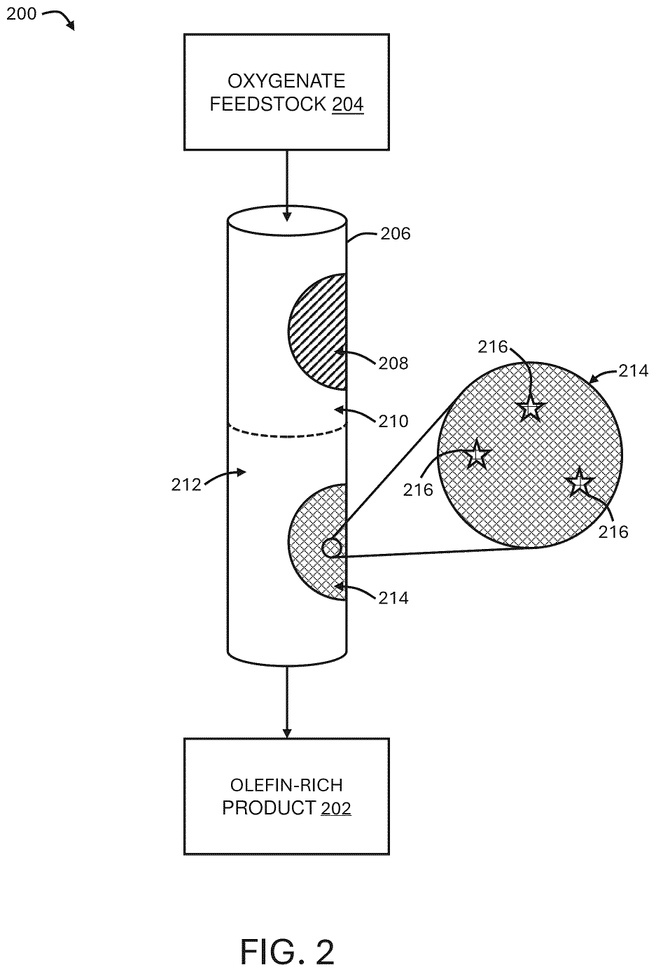

FIG. 2 shows a simplified diagram of another exemplary system 200 for producing an olefin-rich product 202 from an oxygenate feedstock 204 according to additional aspects of the disclosure. Like the system 100 of FIG. 1, the system 200 comprises a reactor 206. In some aspects, the reactor 206 includes a plurality of catalyst materials. For example, the system 200 illustrated in the example of FIG. 2 comprises a tantalum catalyst composition 208 and a second catalyst composition 214. Like the catalyst composition 108 of FIG. 1, the tantalum catalyst composition 208 is configured to convert at least a portion of the oxygenate feedstock 204 into one or more olefins. In some aspects, the second catalyst composition 214 catalyzes conversion of reaction byproducts that emerge from the first catalyst bed 210 (e.g., an ether) into the one or more olefins (e.g., an alkene).

In some aspects, the tantalum catalyst composition 208 is located in a first catalyst bed 210, and the system 200 further comprises a second catalyst bed 212 located downstream of the first catalyst bed 210. The second catalyst bed 212 comprises the second catalyst composition 214. It will also be appreciated that the tantalum catalyst composition 208 and the second catalyst composition 214 can be combined in a mixed catalyst bed.

As introduced above, in some aspects, the second catalyst composition 214 can serve as a mop-up catalyst, converting reaction byproducts that emerge from the first catalyst bed 210 (e.g., an ether) into the one or more olefins (e.g., an alkene). This can result in greater conversion of the oxygenate feedstock 204 into the one or more olefins relative to using the tantalum catalyst composition 208 alone. In some aspects, the second catalyst composition 214 comprises Al. Suitable materials for use as the second catalyst composition 214 include silica, an Al-containing zeolite; a combination of silica and alumina; alumina; and metallic Al. In some aspects, the second catalyst composition 214 further comprises a second metal dopant 216. The second metal dopant 216 can be the same as, or different than, the metal dopant 118 of FIG. 1. In some aspects, the second metal dopant 216 comprises lanthanum. The presence of the second metal dopant 216 can, in some aspects, increase a conversion rate of the second catalyst composition relative to an undoped catalyst composition.

When included, the second metal dopant 216 is, in some aspects, provided in an amount ranging from 0.25 wt. % to 30 wt. % based on total weight of the second catalyst composition. In some aspects, the amount of the second metal dopant 216 ranges from 1 wt. % to 20 wt. % based on total weight of the second catalyst composition. In some aspects, the amount of the second metal dopant 216 ranges from 4 wt. % to 5 wt. % based on total weight of the second catalyst composition.

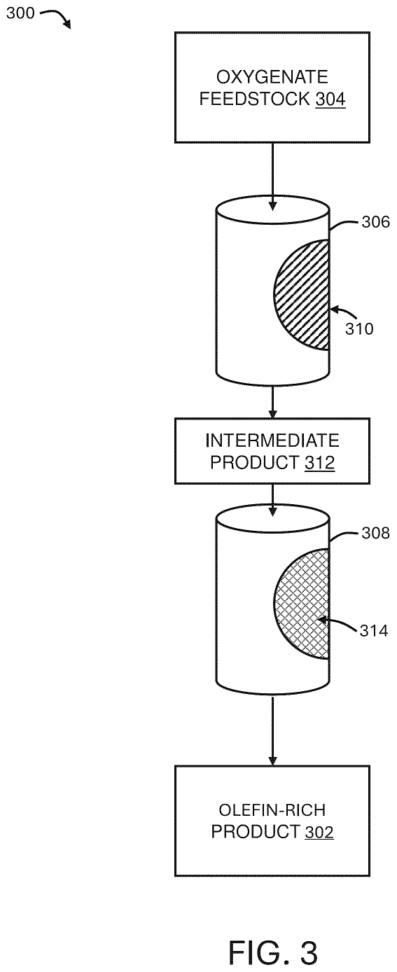

With reference now to FIG. 3, in some aspects, a tantalum catalyst composition and a second catalyst composition can be implemented in separate reactors. FIG. 3 shows a simplified diagram of another exemplary system 300 for producing an olefin-rich product 302 from an oxygenate feedstock 304. The system 300 comprises a first reactor 306 and a second reactor 308. In some aspects, the first reactor 306 comprises a tantalum catalyst composition 310. Like the tantalum catalyst composition 208 of FIG. 2, the tantalum catalyst composition 310 of FIG. 3 is configured to convert at least a portion of the oxygenate feedstock 304 into an intermediate product 312, which is rich in olefins. In some aspects, the intermediate product 312 also includes at least a portion of unreacted oxygenate feedstock and/or one or more byproducts that emerge from the first reactor 306.

As illustrated in FIG. 3, the intermediate product 312 exits the first reactor 306 and enters the second reactor 308. The second reactor 308 contains a second catalyst composition 314. In some aspects, and like the second catalyst composition 214 of FIG. 2, the second catalyst composition 314 of FIG. 3 is configured to convert at least a portion of the intermediate product 312 into the desired olefin-rich product 302. In this manner, the two-reactor system 300 allows for enhanced conversion efficiency and selectivity of the olefin-rich product 302.

### VI. Method of Making a Tantalum Catalyst Composition

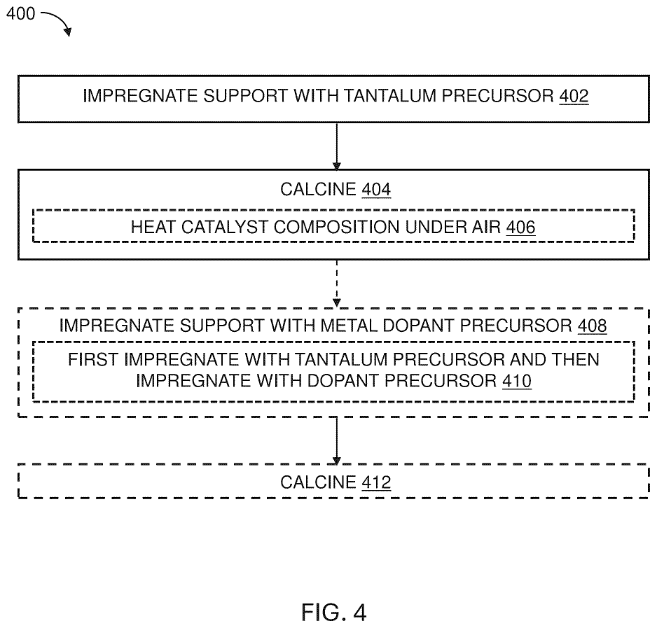

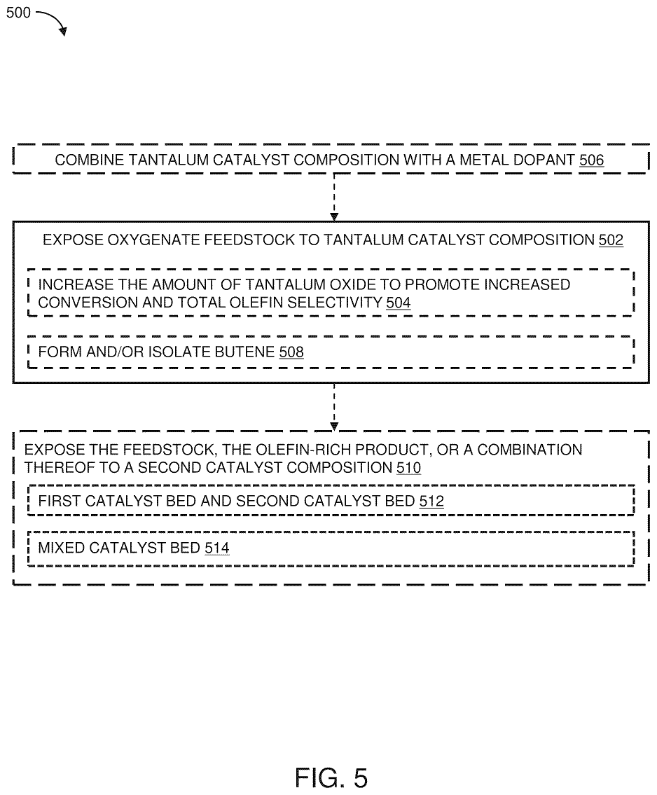

FIG. 4 shows a flow diagram depicting aspects of an exemplary method 400 for making a tantalum catalyst composition for converting an oxygenate feedstock into an olefin-rich product according to aspects of the present disclosure. The following description of the method 400 is provided with reference to FIGS. 1-3 (discussed above) and FIGS. 5-14 (discussed below). It will be appreciated that the method 400 also can be performed in other contexts with the benefit of the present disclosure.

At step 402 of FIG. 4, the method 400 comprises impregnating a support comprising (i) aluminum and/or silicon, and (ii) oxygen, with a tantalum precursor to form a catalyst precursor. Impregnation can refer to co-impregnation or sequential impregnation. Some methods suitable for preparing the catalyst include incipient wetness impregnation, precipitation (including co-precipitation or sequential precipitation), and a sol-gel method. In some aspects, the tantalum precursor comprises Ta (V). Some examples of suitable tantalum precursors include tantalum oxides (e.g., tantalum ethoxide) and tantalum halides (e.g., tantalum chloride, tantalum bromide, tantalum fluoride, tantalum iodide, or any combination thereof). As described in more detail below, calcining the tantalum ethoxide results in oxidation of the ethoxide ligands on the tantalum precursor to form a tantalum oxide (e.g., Ta₂O₅).

At step 404 the method 400 can further comprise calcining the catalyst precursor to form the tantalum catalyst composition. In some aspects, at step 406, calcining the catalyst precursor comprises heating the catalyst precursor under air. As described in more detail below, calcining the catalyst precursor under air can result in greater overall conversion and selectivity than other methods of calcination. In some aspects, calcination can occur under flowing air, static air, under an oxygen-enriched atmosphere, under a NO-containing atmosphere, or a combination thereof.

In some aspects, at step 408, the method 400 further comprises impregnating the support with a second precursor material containing at least one metal dopant. For example, as introduced above, the support can be impregnated with Cr, Zn, Cu, Ag, or a combination thereof. One example of a suitable second precursor material is Cu(NO₃)₂·XH₂O, which introduces Cu into the tantalum catalyst composition.

As indicated at step 410, in some aspects, the support is first impregnated with the tantalum precursor, calcined, and then impregnated with the second precursor material using a sequential impregnation method. In some aspects, and as indicated at 412, the tantalum catalyst composition is calcined again after being impregnated with the second precursor material.

In some aspects, the support is impregnated with the tantalum precursor and the second precursor material using a co-impregnation method. In some aspects, co-impregnation can provide suitable conversion and/or selectivity results. However, in some certain aspects, sequential impregnation can result in greater overall conversion and selectivity as compared to a co-impregnation method.

### VII. Method of Forming an Olefin-Rich Product

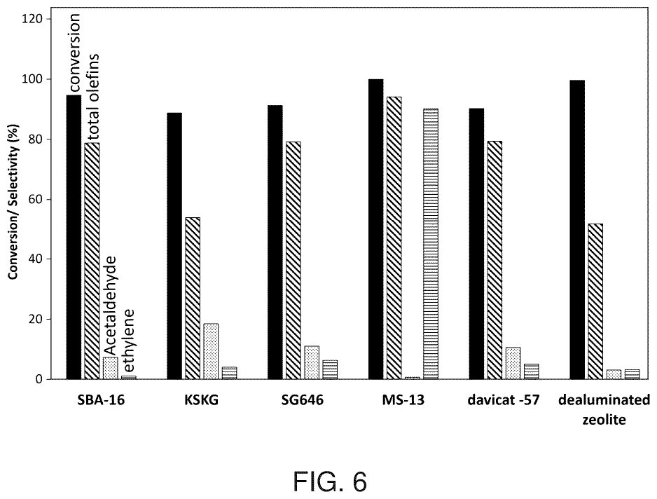

FIG. 5 shows a flow diagram depicting aspects of an exemplary method 500 for exposing an oxygenate feedstock to a tantalum catalyst composition to form an olefin-rich product according to aspects of the present disclosure. The following description of the method 500 is provided with reference to FIGS. 1-4 (discussed above) and FIGS. 6-14 (discussed below). It will be appreciated that the method 500 also can be performed in other contexts with the benefit of the present disclosure.

With reference to FIG. 5, at step 502, the method 500 comprises exposing an oxygenate feedstock to a tantalum catalyst composition, wherein the tantalum catalyst composition comprises tantalum and a support comprising (i) aluminum and/or silicon, and (ii) oxygen. In some aspects, the oxygenate feedstock comprises a C₂-C₄ alcohol, a C₂-C₄ ether, a C₂-C₄ aldehyde, a C₂-C₄ ketone, a C₂-C₄ carboxylic acid, a C₂-C₄ ester, or any combination thereof. In some aspects, the oxygenate feedstock comprises ethanol, acetaldehyde, ethyl acetate, diethyl ether, butyraldehyde, butanol, crotonaldehyde, crotyl alcohol, or a combination thereof.

In some aspects, the tantalum catalyst composition comprises a tantalum oxide in an amount ranging from 1 wt. % to 50 wt. % based on the total weight of the tantalum catalyst composition. In some aspects, at step 504, the method further comprises increasing the amount of tantalum oxide to promote increased conversion and total olefin selectivity, wherein the tantalum catalyst composition comprises Ta₂O₅. In some aspects, the support is present in an amount ranging from 10 wt. % to 90 wt. % based on the total weight of the tantalum catalyst composition.

In some aspects, the feedstock is exposed to the tantalum catalyst composition at a temperature ranging from 200° C. to 450° C. In some aspects, temperatures in the range of 200° C. to 450° C. can facilitate sufficiently activating the tantalum catalyst composition to catalyze the desired chemical reactions, while balancing the reaction rate and catalyst stability, ensuring efficient production while avoiding substantial catalyst degradation and/or unwanted by-product formation. For example, in some aspects, the feedstock is exposed to the tantalum catalyst composition at a temperature ranging from 325° C. to 400° C. In some aspects, operating within the temperature ranges described herein can help to achieve high selectivity towards desired olefin products, such as butene.

In some aspects, the feedstock is exposed to the tantalum catalyst composition at a pressure ranging from ambient atmospheric pressure to 300 psig. In some aspects, the pressure ranges from 35 psig to 100 psig. In some aspects, the pressure ranges from 35 psig to 50 psig. In some certain aspects, pressures above 50 psig may result in the olefin-rich product containing more alkanes and less olefins than when the feedstock is exposed to the tantalum catalyst composition at pressures of 50 psig and below.

In some aspects, the feedstock is exposed to the tantalum catalyst composition at a WHSV ranging from 0.1 hour⁻¹ to 2 hours 1. In some aspects, the feedstock is exposed to the tantalum catalyst composition at a weight hourly space velocity ranging from 0.2 hour⁻¹ to 1 hours⁻¹.

In some aspects, such as at step 506 of FIG. 5, the method 500 comprises combining the tantalum catalyst composition with a metal dopant. In some aspects, the metal dopant comprises a Group 6, Group 11, or Group 12 transition metal. In some aspects, the metal dopant is selected from Cr, Zn, Cu, Ag, or a combination thereof.

In some aspects, as indicated at 508, forming the olefin-rich product comprises forming and/or isolating butene, wherein in, in some aspects, butene selectivity is greater than or equal to 50%. Additional aspects of the conversion rate and selectivity of the tantalum catalyst composition are described in more detail below.

In some aspects and as indicated at step 510, the method further comprises exposing the olefin-rich product, unconverted feedstock, or a combination thereof to a second catalyst composition that catalyzes the formation of additional olefin-rich product. For example, the intermediate product 312 resulting from reaction of the oxygenate feedstock 304 with the tantalum catalyst composition 310 of FIG. 3 can be exposed to the second catalyst composition 314 to generate a further-enriched product with higher olefin content.

As described above and as indicated at 512 of FIG. 5, in some aspects, the oxygenate feedstock is exposed to the tantalum catalyst composition in a first catalyst bed to form an intermediate composition, and the intermediate composition is exposed to the second catalyst composition in a second catalyst bed. In some aspects, such as illustrated at 514 of FIG. 5, the oxygenate feedstock is exposed to the tantalum catalyst composition and the second catalyst composition in a mixed catalyst bed that comprises the tantalum catalyst composition and the second catalyst composition. As described above, the second catalyst composition can serve as a mop-up catalyst that can increase the overall conversion and/or the selectivity of a catalyst system relative to the use of a single tantalum catalyst composition alone.

### VIII. Overview of Several Embodiments

Disclosed herein are aspects of a method, comprising: exposing an oxygenate feedstock to a tantalum catalyst composition to form an olefin-rich product, the catalyst composition comprising tantalum and a support comprising (i) aluminum and/or silicon, and (ii) oxygen.

In any or all of the above aspects, the tantalum catalyst composition comprises a tantalum oxide in an amount ranging from 1% to 50% based on total weight of the tantalum catalyst composition.

In any or all of the above aspects, the support is present in an amount ranging from 10% to 90% based on total weight of the tantalum catalyst composition.

In any or all of the above aspects, the feedstock is exposed to the tantalum catalyst composition at a temperature ranging from 325° C. to 400° C.

In any or all of the above aspects, the method further comprises exposing the feedstock, the olefin-rich product, or a combination thereof to a second catalyst composition that catalyzes formation of additional olefin-rich product.

In any or all of the above aspects, the oxygenate feedstock is exposed to the tantalum catalyst composition in a first catalyst bed to form an intermediate composition, and the intermediate composition is exposed to the second catalyst composition in a second catalyst bed.

In any or all of the above aspects, the oxygenate feedstock is exposed to the tantalum catalyst composition and the second catalyst composition in a mixed catalyst bed that comprises the tantalum catalyst composition and the second catalyst composition.

In any or all of the above aspects, the oxygenate feedstock comprises a C₂-C₄ alcohol, a C₂-C₄ ether, a C₂-C₄ aldehyde, a C₂-C₄ ketone, a C₂-C₄ carboxylic acid, a C₂-C₄ ester, or a combination thereof.

In any or all of the above aspects, the oxygenate feedstock comprises ethanol, acetaldehyde, ethyl acetate, diethyl ether, butyraldehyde, butanol, crotonaldehyde, crotyl alcohol or a combination thereof.

In any or all of the above aspects, the tantalum catalyst composition further comprises a metal dopant.

In any or all of the above aspects, the metal dopant comprises a Group 6, Group 11, or Group 12 transition metal.

In any or all of the above aspects, the metal dopant is selected from Cr, Zn, Cu, Ag, or a combination thereof.

In any or all of the above aspects, forming the olefin-rich product comprises forming butene with a selectivity of greater than or equal to 50%.

In any or all of the above aspects, the tantalum catalyst composition comprises Ta₂O₅, the method further comprising increasing the amount of tantalum oxide to promote increased conversion and total olefin selectivity.

Also disclosed herein are aspects of a method of making a tantalum catalyst composition for converting an oxygenate feedstock into an olefin-rich product, the method comprising: impregnating a support comprising (i) aluminum and/or silicon, and (ii) oxygen, with a tantalum precursor to form a catalyst precursor.

In any or all of the above aspects, the method further comprises calcining the catalyst precursor to form the tantalum catalyst composition.

In any or all of the above aspects, calcining the tantalum catalyst precursor comprises heating the tantalum catalyst precursor under air.

In any or all of the above aspects, the method further comprises impregnating the support with a second precursor material containing at least one metal dopant.

In any or all of the above aspects, the support is first impregnated with the tantalum precursor and then impregnated with the second precursor material.

Also disclosed herein are aspects of a tantalum catalyst composition for converting an oxygenate feedstock into an olefin-rich product, the tantalum catalyst composition comprising: a tantalum oxide in an amount ranging from 1% to 50% based on total weight of the tantalum catalyst composition; and a support comprising aluminum and/or silicon.

### IX: Examples

Aspects of the present teachings can be further understood in light of the following examples.

### Example 1—Catalytic Performance of Cu/Ta₂O₅ Catalysts Supported on Silicon-Based Materials

A series of six 4% Cu/6% Ta₂O₅ catalysts supported on silicon containing materials were synthesized by impregnation of SBA-16 mesoporous silica (ACS Materials), KSKG GOST 3956-76 silica gel, DAVISIL® 646 silica gel, GRACE MS13 silica/alumina, DAVICAT® 57 silica gel, and a dealuminated zeolite. All catalysts were synthesized by sequential impregnation with Ta₂(OC₂H₅)₁₀ first and then Cu(NO₃)₂·XH₂O precursor. Catalysts were calcined with static air. All catalysts were tested under the same operating conditions (temperature=350° C., P=50 psig, WHSVₑₜₕₐₙₒₗ=0.47 hour⁻¹). The H₂/ethanol ratio is equal to 90/10 (molar), with a TOS of 48 hours. Conversion and selectivities toward total olefins (C₂-C₈), acetaldehyde and ethylene are shown in FIG. 6. For all the catalysts the conversion is high and above 85%. Differences in selectivity are observed as summarized in FIG. 6.

### Example 2—Catalytic Performance of Cu/Ta₂O₅ Catalysts Supported on Silicon-Based Materials

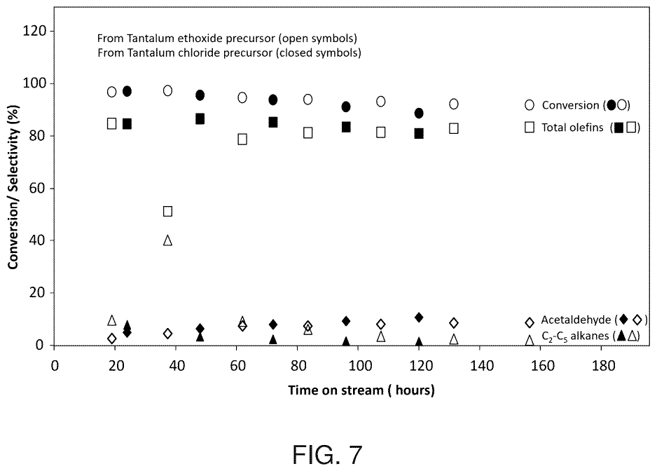

Two catalysts comprising 4% Cu/6% Ta₂O₅ supported on SBA-16 silica were synthesized by sequential impregnation of SBA-16 with first a tantalum precursor and then with copper nitrate hydrate precursor. To understand the impact of the nature of the tantalum precursor on the catalytic performance, one catalyst was prepared using a Ta₂(OC₂H₅)₁₀ precursor and the other one was synthesized with a TaCl₅ precursor. As illustrated in FIG. 7, the conversion and selectivities are similar for both catalysts. All catalysts were synthesized by sequential impregnation with the tantalum precursor first and then Cu(NO₃)₂·XH₂O. Catalysts were calcined with static air. Reactions took place at a temperature of 350° C., pressure of 3.44×10⁵ Pa, WHSVₑₜₕₐₙₒₗ of 0.47 hour⁻¹, and H₂/ethanol ratio equal to 90/10 (molar).

### Example 3—Impact of the Calcination Method on the Catalytic Performance of Cu/Ta₂O₅/SiO₂ Catalysts

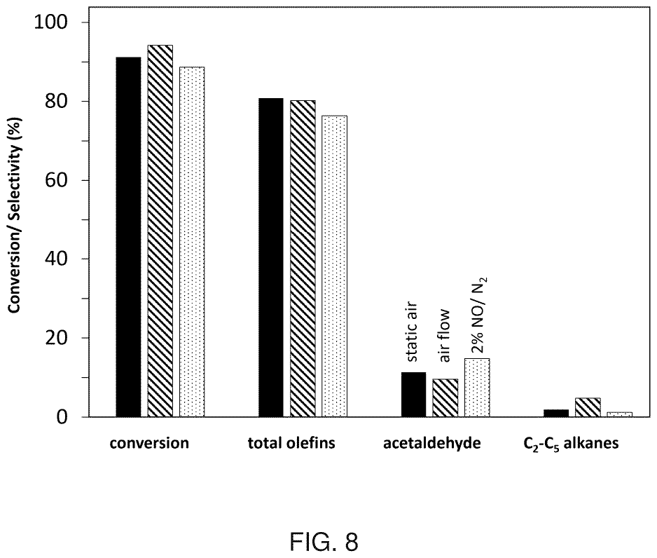

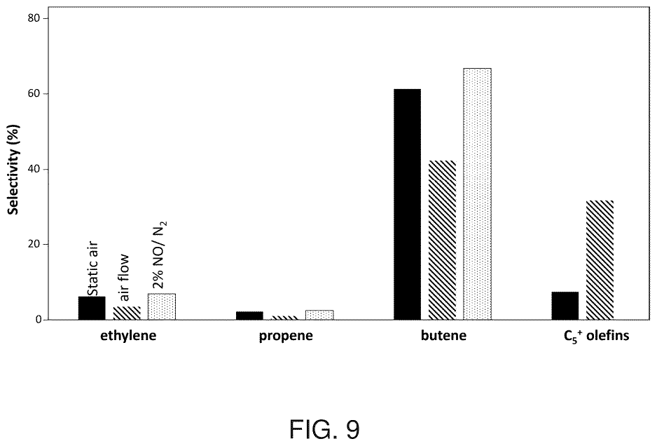

To determine the impact of the calcination method a 4% Cu/6% Ta₂O₅ supported on silica gel, DAVISIL® 646 was treated at 500° C. under either static air, air flow or 2% NO/N₂. The catalytic performance of the three resulting catalysts was tested under the same operating conditions for 48 hours. Conversion and selectivities toward total olefins, acetaldehyde and C₂-C₅ alkanes are shown in FIG. 8. At similar conversion (88-94%), the selectivity toward the total olefins is slightly lower for the catalyst treated under 2% NO/H₂ as compared to those treated under air. As shown in FIG. 9, the selectivities toward butene and C₅ or larger olefins vary depending on the air treatment (i.e., static or flow). Indeed, treatment under air flow favors the formation of olefins with higher carbon numbers as compared to when the catalyst is calcined under static air. All catalysts were synthesized by sequential impregnation with tantalum ethoxide first and then copper nitrate hydrate precursor. All reactions took place at a temperature of 350° C., pressure of 3.44×10⁵ Pa, WHSVₑₜₕₐₙₒₗ of 0.47 hour 1, H₂/ethanol ratio equal to 90/10 (molar), and TOS of 48 hours.

### Example 4—Determination of the Effect of the Method of Preparation (Co-Impregnation Versus Sequential Impregnation) on the Catalytic Performance of Cu/Ta₂O₅/SiO₂ Catalysts

The impact of the method of preparation (i.e., sequential versus co-impregnation) on the catalytic performance was studied for a series of three catalysts comprising 4% Cu and 6% Ta₂O₅. One catalyst was synthesized by co-impregnation of a solution of Cu(NO₃)₂·XH₂O and a solution of Ta₂(OC₂H₅)₁₀. Two catalysts were synthesized by sequential impregnation with either Ta first and then Cu (Ta→Cu) or Cu first and then Ta (Cu→Ta). The results shown in Table 1 indicate that, according to the evaluated examples, higher conversion and total olefins selectivity can be obtained by sequential impregnation of Ta first and then Cu. All reactions took place at a temperature of 350° C., pressure of 3.44×10⁵ Pa, WHSVₑₜₕₐₙₒₗ of 0.47 hour⁻¹, H₂/ethanol ratio equal to 90/10 (molar), and TOS of 48 hours. The Ta₂O₅ precursor is tantalum ethoxide. Catalyst precursors were calcined with static air. Heavy products (e.g., dienes, cyclic paraffins, aromatics) were not detected. Products indicated as “other oxygenates” include 1-butanol, acetic acid, ethyl acetate, crotonaldehyde, acetone and CO₂.

TABLE 1
Catalytic performance of 4% Cu/6% Ta₂O₅ supported on silica SBA-16 prepared by sequential impregnation and co-impregnation.

| | | Selectivity % (carbon based) | | | | | | | | | |
|---|---|---|---|---|---|---|---|---|---|---|---|
| Method of | Conversion | | | | Total | C₂-C₅ | | | | | Other |
| preparation | (%) | Ethylene | Propylene | Butene | olefins | alkanes | Acetaldehyde | Butyraldehyde | DEE | BD | oxygenates |
| Co- | 83.6 | 9.4 | 1.9 | 53.0 | 66.0 | 5.0 | 21.4 | 1.3 | 3.7 | 0.0 | 2.6 |
| impregnation | | | | | | | | | | | |
| Sequential | 94.6 | 6.7 | 2.4 | 63.8 | 76.8 | 8.8 | 7.1 | 0.1 | 5.0 | 0.0 | 2.2 |
| Ta → Cu | | | | | | | | | | | |
| Sequential | 71.1 | 21.2 | 3.0 | 42.9 | 64.0 | 2.2 | 13.3 | 1.6 | 11.7 | 3.1 | 4.1 |
| Cu → Ta | | | | | | | | | | | |

### Example 5—Effect of the Ta₂O₅ Content on the Catalytic Performance

For a catalyst supported on silica SBA-16 and containing 4 wt. % Cu, with Ta₂O₅ content varying from 1.5 wt. % to 10 wt. %, the conversion and selectivity toward different products are shown in Table 2 below. With the increase of Ta₂O₅ loading, the conversion and the total olefin selectivity increase respectively from 79.3 to 94.4% and from 58.5 to 82.4%. Both selectivities to alkanes and DEE increase also with the increase of the Ta₂O₅ loading. This is balanced by a decrease in both acetaldehyde and butyraldehyde selectivities from 29.8 to 5.7% and 1.5 to 0%, respectively. All reactions took place at a temperature of 350° C., pressure of 3.44×10⁵ Pa, WHSVₑₜₕₐₙₒₗ of 0.47 hour⁻¹, H₂/ethanol ratio equal to 90/10 (molar), and TOS of 120 hours. The Ta₂O₅ precursor is Ta₂(OC₂H₅)₁₀. Ta and Cu were co-impregnated on SBA-16 and calcined with static air. Heavy products (e.g., dienes, cyclic paraffins, aromatics) were not detected. Products indicated as “other oxygenates” include 1-butanol, acetic acid, ethyl acetate, crotonaldehyde, acetone and CO₂.

TABLE 2
Catalytic performance of 4% Cu/X % Ta₂O₅/SBA-16 with Ta₂O₅ content (X) varying from 1.5 to 10 wt. %.

| Ta₂O₅ | | Selectivity % (carbon based) | | | | | | | | |
|---|---|---|---|---|---|---|---|---|---|---|
| content | Conversion | | | | Total | C₁-C₅ | | | | Other |
| (wt. %) | (%) | Ethylene | Propylene | Butene | olefins | alkanes | Acetaldehyde | Butyraldehyde | DEE | oxygenates* |
| 1.5 | 79.3 | 3.5 | 0.3 | 54.7 | 58.5 | 0.5 | 29.8 | 1.5 | 2.9 | 6.8 |
| 3 | 87.4 | 6.1 | 1.9 | 72.2 | 80.2 | 0.7 | 13.9 | 0.4 | 3.0 | 1.8 |
| 5 | 88.7 | 10 | 2.3 | 69.3 | 81.6 | 1.5 | 10.7 | 0 | 5.3 | 0.9 |
| 7 | 92.9 | 10.1 | 2.5 | 70.8 | 83.4 | 3.3 | 7.5 | 0.1 | 4.7 | 1.0 |
| 10 | 94.4 | 16.3 | 2.4 | 63.7 | 82.4 | 5.8 | 5.7 | 0 | 5.8 | 0.3 |

### Example 6—Effect of the Cu Loading on the Catalytic Performance of Ta₂O₅/SiO₂

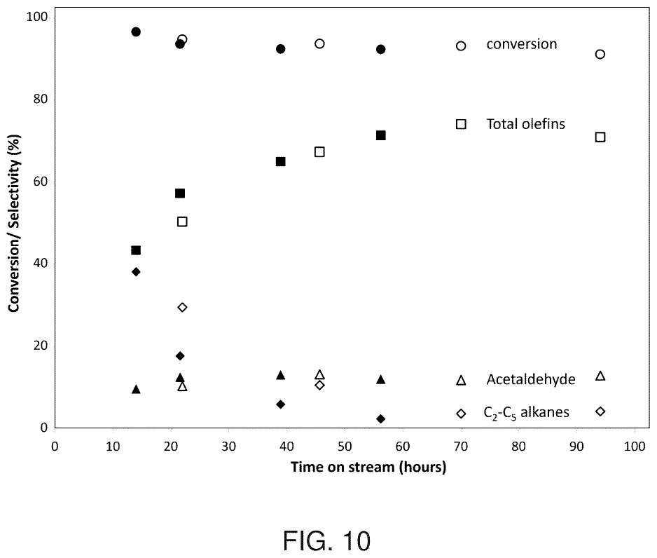

The catalytic performance of an 8% Cu/20% Ta₂O₅ catalyst and a 13% Cu/20% Ta₂O₅ catalyst, both supported on DAVISIL® 646 silica gel, are illustrated in a plot shown in FIG. 10. For both catalysts, the stability profile is similar. Cu loading (12 wt. % vs 13 wt. %) does not appear to have a significant impact on the catalytic activity. Both catalysts were synthesized by sequential impregnation of Ta₂(OC₂H₅)₁₀ followed by Cu(NO₃)₂·XH₂O, and calcined with static air. All reactions took place at a temperature of 350° C., pressure of 3.44×10⁵ Pa, WHSVₑₜₕₐₙₒₗ of 1.88 hour⁻¹, and a H₂/ethanol ratio equal to 82/18 (molar).

### Example 7—Catalyst Performance of 8% Cu/18% Ta₂O₅/SiO₂ Promoted with Elements from Period Four (e.g., Cr, Zn)

To determine the impact of period four elements on the catalytic performance of Cu/Ta₂O₅/SiO₂ catalysts, 8% Cu/18% Ta₂O₅/SG646 (on DAVISIL® 646 silica gel), 0.8% Cr/8% Cu/18% Ta₂O₅/SG646, and 0.8% Zn/8% Cu/18% Ta₂O₅/SG646 catalysts were tested under the same operating conditions. As shown in Table 3, the three catalysts present similar conversion of 93-96%, but differences in selectivities are observed. The catalysts promoted with Zn and Zr present lower selectivity to ethylene but higher selectivity to acetaldehyde, butene and butyraldehyde are obtained. The selectivity to heavy products (e.g., dienes, cyclic paraffins, aromatics) is significantly reduced by addition of Zn and Cr. All reactions took place at a temperature of 350° C., pressure of 3.44×10⁵ Pa, WHSVₑₜₕₐₙₒₗ of 0.47 hour⁻¹, H₂/ethanol ratio equal to 82/18 (molar), and TOS of 190 hours. The Ta₂O₅ precursor is Ta₂(OC₂H₅)₁₀. Ta and Cu were impregnated sequentially on DAVISIL® 646 silica gel with the Ta₂(OC₂H₅)₁₀ precursor first and then the Cu(NO₃)₂·XH₂O precursor. Products identified as “heavies” include dienes, cyclic paraffins, and aromatics. Other oxygenates include 1-butanol, acetic acid, ethyl acetate, crotonaldehyde, acetone and CO₂.

TABLE 3
Comparison of the catalytic performance of 8% Cu/18% Ta₂O₅/SG646,
0.8% Cr/8% Cu/18% Ta₂O₅/SG646 and 0.8% Zn/8% Cu/18% Ta₂O₅/SG646

| | | Selectivity % (carbon based) | | | | | | | | | |
|---|---|---|---|---|---|---|---|---|---|---|---|
| | Conversion | | | | Total | C₂-C₅ | | | | | Other |
| Catalyst | (%) | ethylene | propylene | butene | olefins | alkanes | heavies | acetaldehyde | butyraldehyde | DEE | oxygenates |
| 8% Cu/18% | 94.3 | 13.8 | 3.9 | 59.5 | 82.3 | 1.9 | 4.6 | 3.7 | 0.2 | 4.1 | 3.2 |
| Ta₂O₅/SG646 | | | | | | | | | | | |
| 0.8% Cr/8% | 95.6 | 9.3 | 1.7 | 62.3 | 84.1 | 2.7 | 0.5 | 5.2 | 0.5 | 4.3 | 2.7 |
| Cu/18% | | | | | | | | | | | |
| Ta₂O₅/SG646 | | | | | | | | | | | |
| 0.8% Zn/8% | 93.3 | 8.9 | 1.6 | 59.4 | 72.5 | 1.6 | 0.8 | 13.8 | 0.6 | 7.4 | 3.3 |
| Cu/18% | | | | | | | | | | | |
| Ta₂O₅/SG646 | | | | | | | | | | | |

### Example 8—Catalyst Performance of 4% Cu/6% Ta₂O₅/SG646 with Varied Oxygenated Feedstocks

Feedstock flexibility was demonstrated using a variety oxygenated compounds including ethanol, acetaldehyde, butyraldehyde and crotonaldehyde. Table 4 presents the conversion and selectivity toward total olefins product over the 4% Cu/6% Ta₂O₅/supported on DAVISIL® 646 silica gel. Production of olefins is observed for each oxygenate feedstock. All reactions took place at a pressure of 3.44×10⁵ Pa and H₂/ethanol ratio equal to 90/10 (molar), and TOS of 48 hours. The Ta₂O₅ precursor is Ta₂(OC₂H₅)₁₀. Ta and Cu were impregnated sequentially on DAVISIL® 646 silica gel with the Ta₂(OC₂H₅)₁₀ precursor first and then the Cu(NO₃)₂·XH₂O precursor.

TABLE 4
Conversion and total olefins selectivity for upgrading
of oxygenates (e.g., ethanol, acetaldehyde, crotonaldehyde,
butyraldehyde) into olefins over 4% Cu/6% Ta₂O₅/supported
on DAVISIL ® 646 silica gel.

| | | Ethanol + | Butyraldehyde + |
|---|---|---|---|
| | | acetaldehyd [symbol] | crotonaldehyde |
| Feedstock | Ethanol | (2/1 weight ratio) | (1/1 weight ratio) |
| Temperature (° C.) | 350 | 350 | 270 |
| WHSV feed (hr⁻¹) | 0.47 | 0.47 | 0.95 |
| Total conversion (%) | 91.2 | 82.6 | >99 |
| Ethanol conversion (%) | 91.2 | 92.3 | N/A |
| Acetaldehyde conversion | N/A | 57.4 | N/A |
| (%) | | | |
| Butyraldehyde | N/A | N/A | >99 |
| conversion (% [symbol] | | | |
| Crotonaldehyde | N/A | N/A | >99 |
| conversion ( [symbol] | | | |
| Total olefins selectivity | 80.8 | 90.5 | 41.5 |
| (%) | | | |

[symbol]

indicates data missing or illegible when filed

### Example 9—Impact of the Addition of Water in the Ethanol Feedstock on the Catalytic Performance of Cu/Ta₂O₅/SiO₂ Catalysts

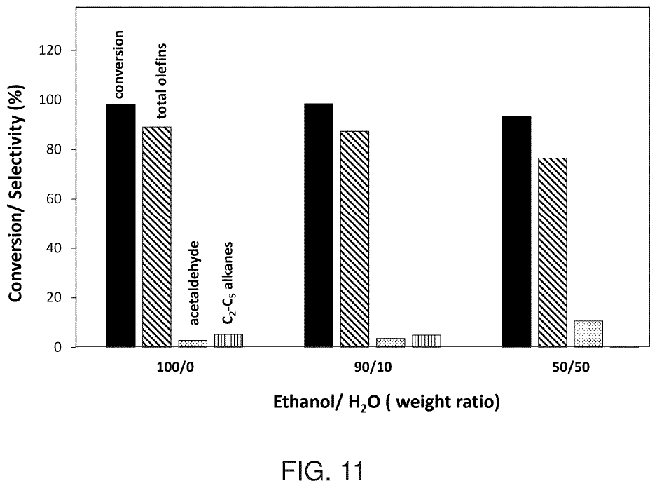

The impact that the presence of H₂O in the feedstock can have on the activity was tested for ethanol/H₂O ratios of 100/0, 90/10 and 50/50 (weight ratio) over a 0.8% Cr/8% Cu/20% Ta₂O₅ catalyst supported on DAVISIL® 646 silica gel. The results presented in FIG. 11 demonstrate that the conversion decreases from 98% to 93.4% with the increase of the water content from 0 to 50 wt. %. Similarly, the total olefin selectivity decreases from 89 to 76.5% and the alkane selectivity decreases from 5.3 to 0.4% with the increase of the water content from 0 to 50 wt. %. This is balanced by an increase of the acetaldehyde selectivity from 2.8 to 10.7%. All catalysts were synthesized by sequential impregnation of Ta₂(OC₂H₅)₁₀ followed by Cu(NO₃)₂·XH₂O and calcined with static air. All reactions took place at a temperature of 350° C., pressure of 3.44×10⁵ Pa, WHSVₑₜₕₐₙₒₗ ranging from 0.4 hour⁻¹-0.5 hour⁻¹, H₂/ethanol ratio equal to 82/18 (molar), and TOS of 190 hours. The 100% ethanol solution had an 82/18 (molar) H₂/ethanol ratio. The H₂/ethanol/H₂O ratio was 78/17/48 (molar) for 90% ethanol/10% water and 50/14/36 (molar) for 50% ethanol/50% water.

### Example 10—Stability Profile for 0.2% Cr/4% Cu/20% Ta₂O₅ Supported on SG646 Silica Gel

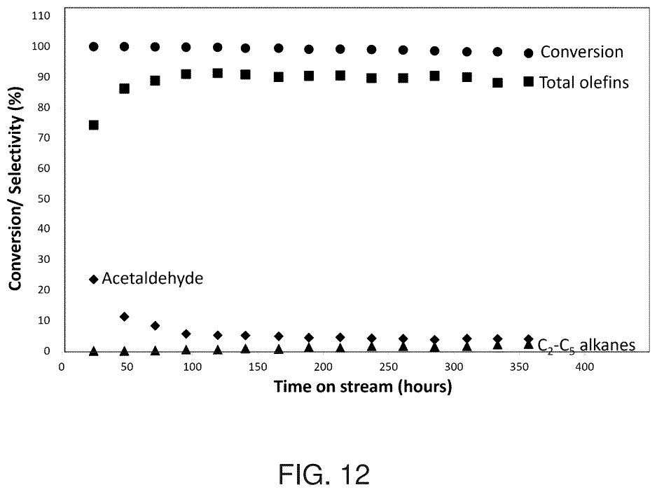

FIG. 12 shows a lifetime study for 0.2% Cr/4% Cu/20% Ta₂O₅ supported on DAVISIL® 646 silica gel. The catalyst shows negligeable loss of conversion for over 350 hours on stream. The selectivity for olefins is high and equal to 88-91% with a TOS of 72 hours to 357 hours. The catalysts were synthesized by sequential impregnation of tantalum ethoxide followed by copper nitrate hydrate precursor and calcined with static air. All reactions took place at a temperature of 350° C., pressure of 3.44×10⁵ Pa, WHSVₑₜₕₐₙₒₗ of 0.24 hour⁻¹, and a H₂/ethanol ratio equal to 82/18 (molar).

### Example 11—Addition of a Second Catalyst to Favor DEE Conversion to Ethylene

Table 5 below shows that catalysts comprising Al and O are active for converting DEE into ethylene. All reactions took place at a pressure of 3.44×10⁵ Pa, a TOS ranging from 33 hours to 48 hours, and a N₂/feed ratio of 95.7/4.3 (molar). The WHSV_{DEE} was 1.69 hr⁻¹ for La/Al₂O₃ and SiO₂—Al₂O₃ and the WHSV_{DEE} was 0.85 hr⁻¹ for USY.

TABLE 5
Catalytic performance of catalysts comprising
Al and O for DEE conversion to olefins.

| | | Selectivity % (carbon based) | | | | |
|---|---|---|---|---|---|---|
| | | | | | | Other |
| Catalyst | Conversion | Ethylene | Butene | Acetaldehyde | Ethane | oxygenates |
| USY | 87.8 | 95.6 | 0.0 | 3.1 | 1.1 | 0.2 |
| La/Al₂O₃ | 95.5 | 95.0 | 2.7 | 1.5 | 0.6 | 0.2 |
| SiO₂-Al₂O | 99.9 | 99.5 | 0.0 | 0.5 | 0.0 | 0.0 |

The catalysts described in Table 5 can be added to the reactor containing the tantalum-based catalyst to help convert DEE produced from ethanol into ethylene in the presence of H₂ without leading to conversion of ethylene into ethane.

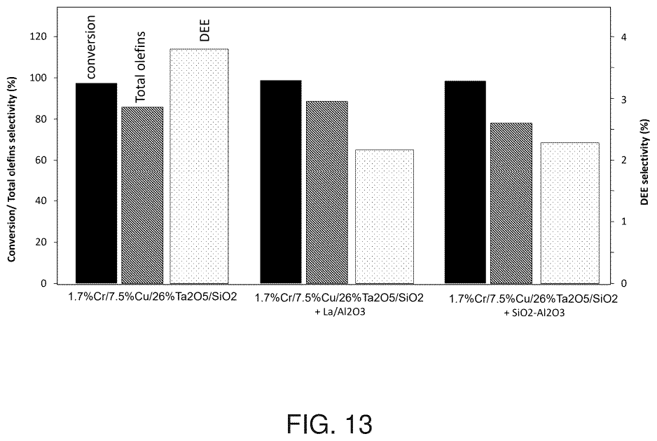

FIG. 13 presents the conversion, total olefins selectivity and the DEE selectivity for one reactor filled with the tantalum-based catalyst alone (1.7% Cr/7.5% Cu/26% Ta₂O₅ supported on DAVICAT® 57 silica gel), for one reactor filled with the tantalum-based catalyst alone (1.7% Cr/7.5% Cu/26% Ta₂O₅ supported on DAVICAT® 57 silica gel) on top of the La/Al₂O₃ catalyst and for one reactor filled with the tantalum-based catalyst alone (1.7% Cr/7.5% Cu/26% Ta₂O₅ supported on DAVICAT® 57 silica gel) on top of the SiO₂—Al₂O₃ catalyst. The DEE selectivity is lower when the reactor is filled with the tantalum-based catalyst and the catalyst containing either La/Al₂O₃ or SiO₂—Al₂O₃. The catalysts were synthesized by sequential impregnation of Ta₂(OC₂H₅)₁₀ followed by Cu(NO₃)₂·XH₂O and calcined with static air. All reactions took place at a temperature of 350° C., pressure of 3.44×10⁵ Pa, H₂ flow of 15 sccm, ethanol flow of 0.012 mL/min, and H₂/ethanol ratio of 76/24 (molar).

### Example 12—Comparison of the Catalytic Performance of Ta-Based Catalyst with Zr-Based Catalyst

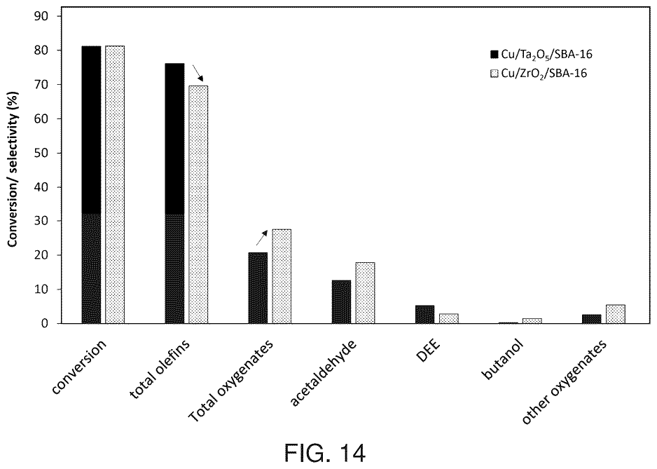

FIG. 14 presents the catalytic performance results for 4% Cu/6% Ta₂O₅ supported on SBA-16 and for 4% Cu/6% ZrO₂ supported on SBA-16 operated under the same reaction conditions. While the conversion is similar for both catalysts, the selectivity to the desired olefins is higher for the tantalum-based catalyst. This is balance by a decrease in selectivity to oxygenates. The tantalum-based catalyst presents acid properties that favor conversion of acetaldehyde, butanol and other intermediates oxygenates (e.g., butyraldehyde) into n-butene. All reactions took place at a temperature of 350° C., pressure of 3.44×10⁵ Pa, WHSVₑₜₕₐₙₒₗ of 0.47 hour⁻¹, and a H₂/ethanol ratio equal to 82/18 (molar). The products identified as “total oxygenates” in FIG. 14 include acetaldehyde, DEE, butanol, and other oxygenates. Other oxygenates include acetic acid, ethyl acetate, methanol, butyraldehyde, crotonaldehyde, acetone and CO₂.

While preferred embodiments of the present disclosure have been shown and described herein, it will be apparent to a person of ordinary skill in the art that such embodiments are provided by way of example only. Variations, changes, and substitutions to these disclosed embodiments will be apparent to a person of ordinary skill in the art without departing from the present disclosure. It should be understood that all such various alternatives to the embodiments described herein may be employed in practicing the present disclosure. The following claims define the scope of the disclosure.

We claim:

1. A method, comprising: exposing an oxygenate feedstock to a tantalum catalyst composition to form an olefin-rich product, the tantalum catalyst composition comprising a support comprising (i) aluminum and/or silicon, and (ii) oxygen.

2. The method of claim 1, wherein the tantalum catalyst composition comprises a tantalum oxide in an amount ranging from 1% to 50% based on total weight of the tantalum catalyst composition.

3. The method of claim 1, wherein the support is present in an amount ranging from 10% to 90% based on total weight of the tantalum catalyst composition.

4. The method of claim 1, wherein the feedstock is exposed to the tantalum catalyst composition at a temperature ranging from 325° C. to 400° C.

5. The method of claim 1, further comprising exposing the feedstock, the olefin-rich product, or a combination thereof to a second catalyst composition that catalyzes formation of additional olefin-rich product.

6. The method of claim 5, wherein the oxygenate feedstock is exposed to the tantalum catalyst composition in a first catalyst bed to form an intermediate composition, and the intermediate composition is exposed to the second catalyst composition in a second catalyst bed.

7. The method of claim 5, wherein the oxygenate feedstock is exposed to the tantalum catalyst composition and the second catalyst composition in a mixed catalyst bed that comprises the tantalum catalyst composition and the second catalyst composition.

8. The method of claim 1, wherein the oxygenate feedstock comprises a C₂-C₄ alcohol, a C₂-C₄ ether, a C₂-C₄ aldehyde, a C₂-C₄ ketone, a C₂-C₄ carboxylic acid, a C₂-C₄ ester, or a combination thereof.

9. The method of claim 1, wherein the oxygenate feedstock comprises ethanol, acetaldehyde, ethyl acetate, diethyl ether, butyraldehyde, butanol, crotonaldehyde, crotyl alcohol or a combination thereof.

10. The method of claim 1, wherein the tantalum catalyst composition further comprises a metal dopant.

11. The method of claim 10, wherein the metal dopant comprises a Group 6, Group 11, or Group 12 transition metal.

12. The method of claim 11, wherein the metal dopant is selected from Cr, Zn, Cu, Ag, or a combination thereof.

13. The method of claim 1, wherein forming the olefin-rich product comprises forming butene with a selectivity of greater than or equal to 50%.

14. The method of claim 1, wherein the tantalum catalyst composition comprises Ta₂O₅, the method further comprising increasing the amount of tantalum oxide to promote increased conversion and total olefin selectivity.

15. A method of making a tantalum catalyst composition for converting an oxygenate feedstock into an olefin-rich product, the method comprising impregnating a support comprising (i) aluminum and/or silicon, and (ii) oxygen, with a tantalum precursor to form a catalyst precursor.

16. The method of claim 15, further comprising calcining the catalyst precursor to form the tantalum catalyst composition.

17. The method of claim 15, further comprising impregnating the support with a second precursor material containing at least one metal dopant.

18. The method of claim 15, wherein calcining the catalyst precursor comprises heating the catalyst precursor under air.

19. The method of claim 16, wherein the support is first impregnated with the tantalum precursor and then impregnated with the second precursor material.

20. A tantalum catalyst composition for converting an oxygenate feedstock into an olefin-rich product, the tantalum catalyst composition comprising:
a tantalum oxide in an amount ranging from 1% to 50% based on total weight of the tantalum catalyst composition; and
a support comprising aluminum and/or silicon.
